(19) United States
(12) Patent Application Publication
(10) Pub. No.: US 2018/0116862 A1
(43) Pub. Date: May 3, 2018

(54) PRE-FORMED PARTIAL COVERAGE OCCLUSAL SPLINT

(71) Applicant: Jean Feldman, Hutto, TX (US)

(72) Inventor: Jean Feldman, Hutto, TX (US)

(21) Appl. No.: 15/801,915

(22) Filed: Nov. 2, 2017

Related U.S. Application Data
(60) Provisional application No. 62/416,500, filed on Nov. 2, 2016.

(51) Int. Cl. A61F 5/56

(52) U.S. Cl. CPC A61F 5/566; A61F 2005/563

(57) ABSTRACT

A partial occlusal splint for the placement on the central and lateral upper or lower portion of a wearer's teeth for the prevention of clenching and grinding. The splint device comprised of a pair of materials having dissimilar durometers and adapted to form a channel for the engagement of the selected teeth of the wearer. A ridge portion is positioned on an exterior of the device opposite the channel and adapted to create a gap to prevent contact of the wearer's teeth during wearing of the device. The channel including at least one aperture. The at least one aperture providing additional surface area for the engagement of a removable securing means. The removable securing means comprised of a resilient and compressible material for securing the device to the teeth.

### CROSS REFERENCE TO RELATED APPLICATIONS

This application claims priority to U.S. Provisional Patent Application No. 62/416,500 filed 2 Nov. 2016 to the above named inventor, and is herein incorporated by reference in its entirety.

### FEDERALLY SPONSORED RESEARCH OR DEVELOPMENT

Not Applicable

### SEQUENCE LISTING, A TABLE, OR A COMPUTER PROGRAM

Not Applicable

### FIELD OF THE INVENTION

The present disclosure relates generally to a pre-formed partial occlusal splint for the treatment of bruxism and designed for usage during the daytime.

### BACKGROUND OF THE INVENTION

Bruxism is commonly known as teeth grinding and clenching. This clenching and grinding can often lead to hyperactivity of the trigeminal nerve. This hyperactivity frequently leads to additional ailments in the form of frequent headaches, including migraine headaches.

One method for the treatment of bruxism is through the use of an intraoral dental device, such as a splint or mouthguard, to generally form a gap or space between the teeth to prevent grinding and relieve tension on the trigeminal nerve.

Traditionally, these intraoral dental devices are formed with a dental office through molding and may be formed specifically for use in the mouth of a given user. Still other devices may be formed through the use a material adapted for heating and molding to the bite of user, generally provided in a material that is described as of the “boil and bite type.” This typical device is most often shaped and adapted to conform specifically to the bite of a wearer and generally formed to conform to the upper teeth of the wearer and extending over and around the posterior teeth.

Although these devices are generally effective for the treatment of bruxism and the hyperactivity of the trigeminal nerve, they typically require an appointment at a dental office or with a dental professional for molding and forming. Further, as the typical splint extends across the entire bite of the upper teeth of a wearer, it can be cumbersome, uncomfortable, and irritating. Still further, these devices are generally constructed in a permanent position wherein the device is not adapted to allow for additional customization or movement, if the wearer of the device has a change in bite. Therefore, there is a need for improved intraoral device for the treatment of bruxism and associated headaches that is primarily designed for daytime use or wake time hours. Preferably, the device is easily formed by a user, without boiling, and specifically designed for placement on the front teeth of a wearer including both the upper or lower teeth.

### SUMMARY OF THE INVENTION

The present disclosure provides an improved device adapted for placement within the mouth of a wearer to generally prevent contact of the upper and lower canines and molars during wearing offering relief from clenching and grinding and hyperactivity of the trigeminal nerve during use. The device of the disclosure is designed for placement on the upper or lower incisors and lateral teeth of the wearer and adapted for placement and fitting without requiring the need to heat the material to allow for proper fit.

The device of the present disclosure is generally U-shaped or J-shaped when viewed from a side profile and forming a channel region for the placement of teeth of the wearer. The device has a slightly arcuate shape to generally conform to the jawline of the wearer. The device is comprised of multiple materials and material types including an exterior surface and an interior surface, with the interior surface generally aligned with an interior of the channel region. The interior surface is generally adapted of a material having a composition that is generally softer than the exterior material. Accordingly, the interior material having a durometer less than the durometer of the exterior material. The exterior material having a durometer greater than the interior material with the durometer having a value to prevent deformation of the exterior portion during use.

A lower portion of the exterior extends opposite the channel region and is generally adapted to extend a height opposite the lower portion to generally form at least one ridge. The at least one ridge adapted as the portion of the exterior surface that is contacted by the opposed teeth engaged within the channel, wherein the gap formed by the device is generally formed by the height of the ridge portion. The at least one ridge placed at a central portion along a width of the device.

The device includes at least one aperture positioned within the channel and extending from the interior to the exterior of the device. The at least one aperture generally provided as an additional surface area to allow for the attachment of a securing means to aid in removably securing the device within the mouth of the wearer. The securing means is comprised of a material that is generally resilient and deformable and adapted to be removed and replaced when desired. Preferably, this securing means is a gum or gum-like material that may be enhanced with a flavoring element. Preferably, this flavoring element is selected from an essential oil.

### BRIEF DESCRIPTION OF THE SEVERAL VIEWS OF THE DRAWING(S)

The accompanying drawings are included to provide a further understanding of the present invention and are incorporated in and constitute a part of this specification. The drawings illustrate exemplary embodiments of the present invention and together with the description serve to further explain the principles of the invention. Other aspects of the invention and the advantages of the invention will be better appreciated as they become better understood by reference to the Detailed Description when considered in conjunction with accompanying drawings, and wherein:

FIG. 1 shows a front side view of the device, according to the present invention;

FIG. 2 shows a top view of the device, according to the present invention;

FIG. 3 shows an isometric view of the device, according to the present invention;

FIG. 4 shows a side view of the device, according to the present invention;

FIG. 5 shows a cross sectional view of the device, according to the present invention; and

FIG. 6 shows an isometric side view with the teeth engaged, according to the present invention.

### DETAILED DESCRIPTION OF THE INVENTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful, and is not intended to exclude other embodiments from the scope of the invention.

As used herein, the term “coupled” means the joining of two members directly or indirectly to one another. Such joining may be stationary in nature or movable in nature and/or such joining may allow for the flow of fluids, electricity, electrical signals, or other types of signals or communication between two members. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

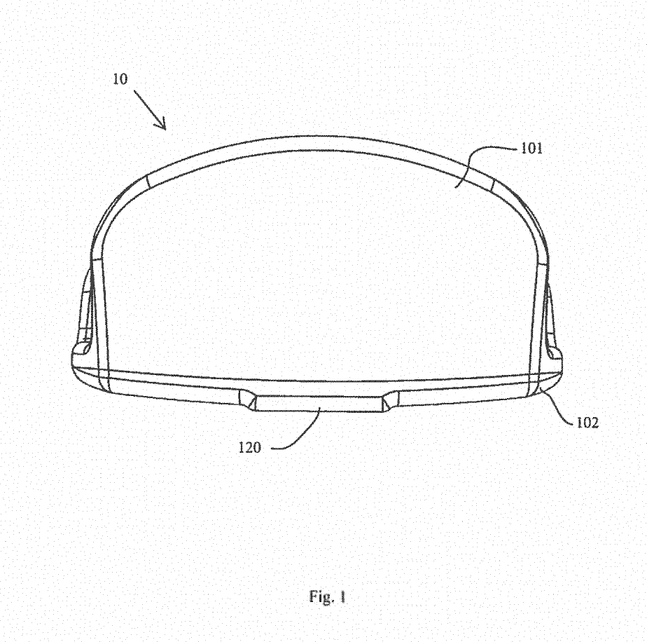

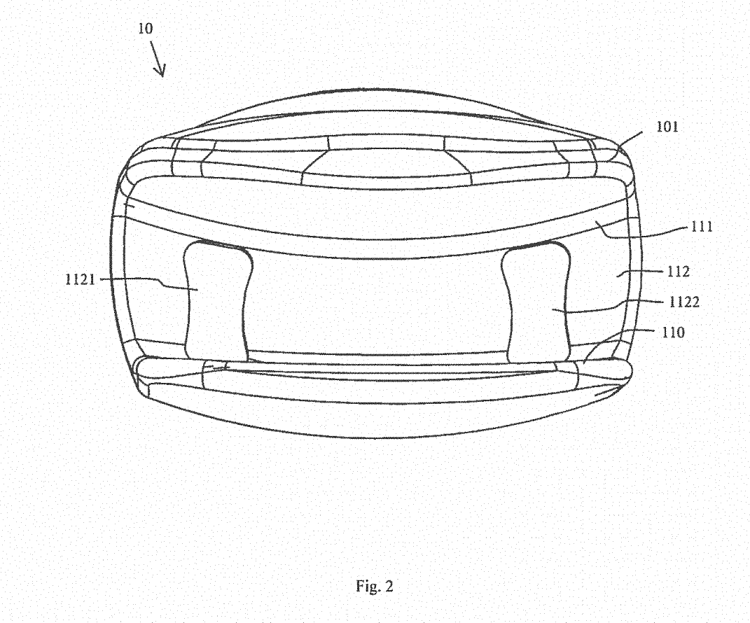

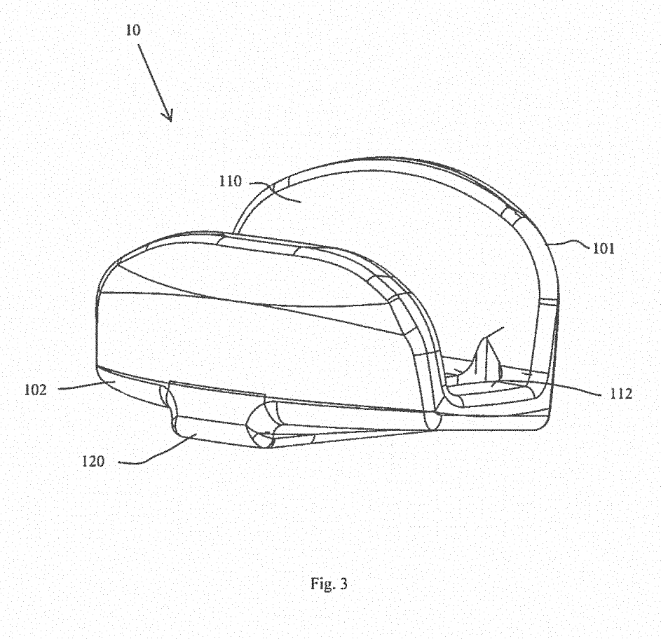

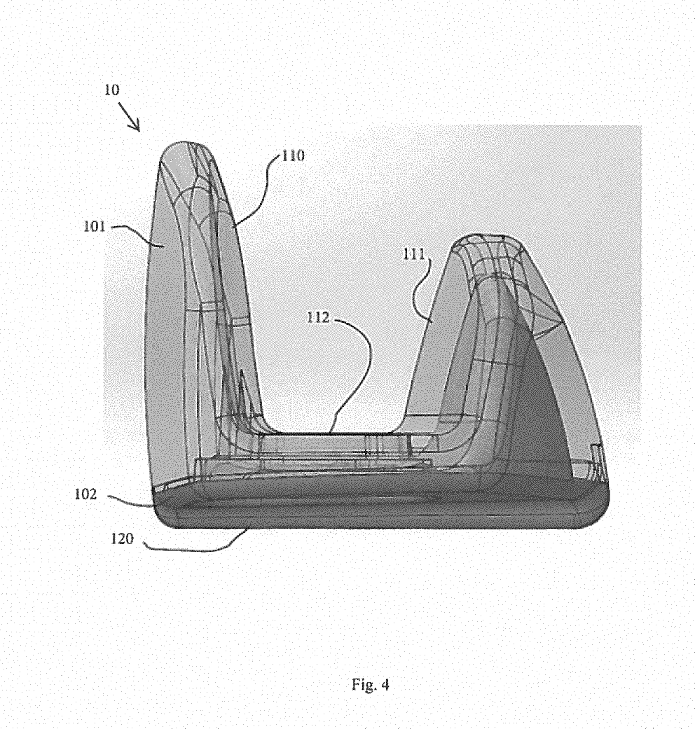

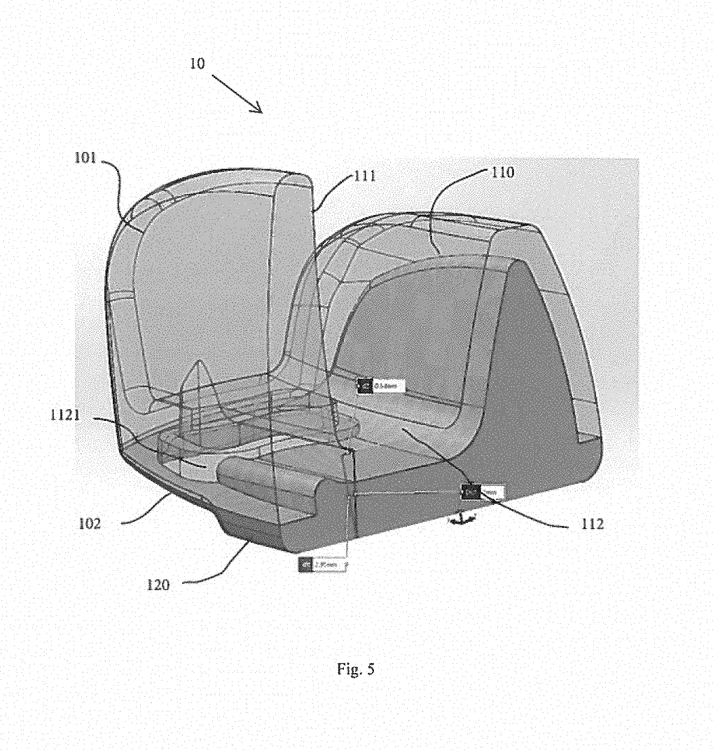

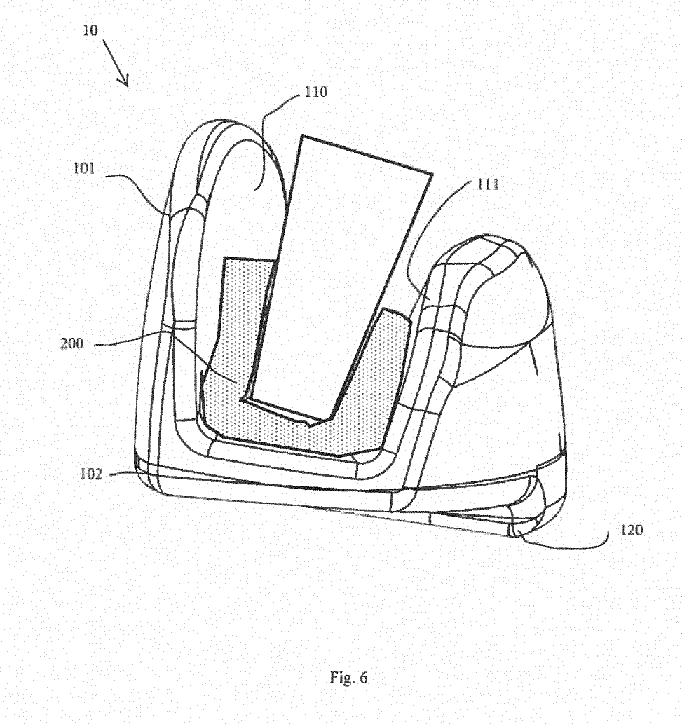

Referring now to FIGS. 1-6, the device of the present disclosure is generally referred to as occlusal splint 10. The splint 10 is generally adapted for the treatment of bruxism and generally adapted to form a gap between the upper and lower teeth to relieve pressure on the trigeminal nerve. The splint 10 is designed for placement on the upper or lower incisors and lateral teeth of wearer and adapted for fitting without the need to heat or otherwise pre-mold the device for proper placement. The device 10 having a slightly arcuate shape to generally conform to the jawline of the wearer.

The occlusal splint device 10 is comprised of a pair of distinct materials forming an interior portion 101 and an exterior portion 102. The interior portion 101 generally U-shaped or J-shaped in cross section with a first interior side 110 opposed a second interior side 111. The first interior side 110 and second interior side 111 comprising the stem portions of the U-shape or J-shape of the device 10 with a space between the first interior side 110 and the second interior side 111 forming a channel 112. The channel 112 allowing for the placement of the incisors and lateral teeth for generally securing the device 10. The channel 112 generally extending a portion of a length of the device defined by the distance between the first side 110 and second side 111 and extending the entire width of the device 10 defined as the distance between opposed edges of the device 10.

The exterior portion 102 positioned at an underside of the interior portion 101 and extending a width and a length of the device 10. The exterior portion 102 including at least one ridge 120. The at least one ridge 120 extending a height opposite the channel 112 and adapted as the portion of the exterior portion 102 that is contacted by the opposed teeth of those teeth engaged within the channel 112, wherein a gap between the upper teeth and the lower teeth of the wearer is created by the at least one ridge portion 120 of the device 10. The distance of the gap generally corresponding to the height of the at least one ridge portion 120. In the preferred embodiment of the present invention, the at least one ridge 120 is placed at a central position along a length of the device and extending a height between 1.5 mm and 2.5 mm with 2 mm as the preferred height of the at least one ridge 120. In an alternate embodiment of the present invention, the device 10 includes a pair of ridges 120, with the pair of ridges 120 generally spaced apart from each other.

The distinct materials of the interior portion 101 and the exterior portion 102 generally selected to provide comfort and durability during use of the splint 10. According, the interior portion 101 is comprised of a material selected to have a material composition that is generally softer than the composition of the material of the exterior portion 102. Accordingly, the interior portion 101 material has a durometer less than a durometer of the exterior portion 102. The exterior portion 102 durometer greater than the interior portion 101 with the durometer of the exterior portion 102 selected to have hardness value to prevent deformation of the exterior portion 102 from biting and clenching during use. Preferably, the exterior portion 102 having a durometer within a range of 60 to 100 as measured on the Shore A hardness scale.

The device 10 includes at least one aperture 1121 positioned within the channel 112 and having a depth extending from the interior portion 101 to the exterior portion 102 of the device 102 The at least one aperture 1121 positioned adjacent to the at least one ridge 120 central position. The at least one aperture 1121 generally provided as additional surface area to allow for the attachment of a securing means 200 to aid in removably securing the device 10 within the mouth of the wearer.

In a preferred embodiment of the present disclosure, the device 10 includes a pair of apertures 1121, 1122, with a first aperture 1121 of the pair of apertures 1121, 1122 positioned on a side of the ridge portion 120 and a second aperture 1122 of the pair of apertures 1121, 1122 positioned on an opposed side of the ridge portion 120 relative to the first aperture 1121.

The securing means 200 is adapted and formulated as a material that is generally resilient and deformable and adapted to be removed and replaced when desired. The securing means 200 is positioned within the channel 112 and engaged within the at least one aperture 1121. Accordingly, the securing means 200 is adapted to form to the bite of the wearer and generally allow for a customized fit of the device 10. The securing means 200 is preferably a gum or gum-like material that may be enhanced with a flavoring element. Preferably, this flavoring element is selected from an essential oil.

In use, a user will generally select the device 10 from the various packaging the device 10 is placed within, add the securing means 200 within the channel 112 in engagement with the at least one aperture 1121, and place the device 10 upon their upper or lower incisors or lateral teeth. The device 10 will generally create a gap between the upper and lower teeth of the wearer and generally relax the wearer's jaw and hyperactivity of the trigeminal nerve.

In the preferred embodiment of the device 10 of the present disclosure, the device 10 materials are preferably comprised of a transparent material, such as transparent plastic material.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) but that the invention will include all embodiments falling with the scope of the appended claims.

What is claimed is:

1. A device adapted for placement in a mouth of a wearer for the treatment of bruxism, the device comprising:
an interior portion, the interior portion having a durometer and a first side and a second side, a distance between the first side and the second side defining a channel;
the channel shaped for the engagement of a portion of the teeth of the wearer, the channel having at least one aperture;
an exterior portion, the exterior portion coupled to the interior portion opposite the channel, the exterior portion having a durometer, the durometer harder than the durometer of the interior portion, the exterior portion having a ridge;
the ridge extending a height from the exterior portion opposite the channel, the ridge extending a length of the device; and
a securing means, the securing means comprised of a resilient material adapted for removable receipt within the channel, wherein the securing means is generally compressible for molding to the teeth of the wearer.

2. The device as in claim 1, wherein the channel includes a pair of apertures.

3. The device as in claim 1, wherein the ridge extends a height between 1.5 mm and 2.5 mm.

4. The device as in claim 1, wherein securing means is a gum.

5. The device as in claim 1, wherein the securing means has a flavor.

6. The device as in claim 5, wherein the flavor is selected from an essential oil.

7. The device as in claim 1, wherein the ridge is positioned at a central portion of the exterior portion.

8. The device as in claim 1, wherein the exterior portion includes a pair of ridges.

9. The device as in claim 1, wherein the exterior portion durometer is between 60 and 100 on the Shore A hardness scale.

10. The device as in claim 1, wherein the exterior portion is comprised of a transparent material.

11. The device as in claim 1, wherein the interior portion is comprised of a transparent material.

12. The device as in claim 11, wherein the exterior portion is comprised of a transparent material.

13. A method for the treatment of bruxism using the device as in claim 1, wherein the device in placed on the central and lateral upper teeth of the wearer.

14. A method for the treatment of bruxism using the device as in claim 1, wherein the device is placed on the central and lateral lower teeth of the wearer.
